(19) United States
(12) Patent Application Publication
(10) Pub. No.: US 2026/0166062 A1
(43) Pub. Date: Jun. 18, 2026

(54) IRRADIATION PROTECTION METHODS, USES AND COMPOSITIONS

(71) Applicant: XERIENT PHARMA LTD., Petah Tikva (IL)

(72) Inventors: Michael Shtein, Potomac, MD (US); Guy Yachin, Potomac, MD (US)

(21) Appl. No.: 19/535,864

(22) Filed: Feb. 10, 2026

Related U.S. Application Data
(63) Continuation-in-part of application No. PCT/US2024/037466, filed on Jul. 10, 2024.
(60) Provisional application No. 63/525,743, filed on Jul. 10, 2023.

(51) Int. Cl. A61K 31/661; A61K 9/00; A61K 9/08; A61K 47/32; A61P 39/06

(52) U.S. Cl. CPC A61K 31/661; A61K 9/0053; A61K 9/08; A61K 47/32; A61P 39/06

(57) ABSTRACT

The disclosure relates to methods and compositions for use in protecting portions of the small intestine. More specifically, the disclosure is related to methods and uses of nasal tubes in targeted delivery of compositions of cytoprotectants to a predetermined location adjacent an organ or a tissue sought to be irradiated in a subject in need thereof.

### BACKGROUND

The disclosure is directed to methods and compositions for use in protecting portions of the small intestine. More specifically, the disclosure is related to methods and uses of nasal tubes in targeted delivery of compositions of radioprotectants to a predetermined location adjacent an organ or a tissue sought to be irradiated in a subject in need thereof.

In certain organs, the major limiting factor in delivering the appropriate tumoricidal dose of ablative radiation is radiation-induced toxicity to normal tissue in adjacent organs or tissues. This issue is underscored in solid tumors of the abdomen and pelvis, such as pancreatic and prostate adenocarcinoma, which often cannot achieve tumoricidal doses without significant morbidity to the gastrointestinal (GI) tract. For example, pancreatic cancer often occurs in the head of the pancreas, which shares blood supply with the duodenum—a radiosensitive portion of the intestinal tract. Tumors of the pancreatic head require doses that exceed about 77 Gy to achieve local control, an often impossible endeavor to administer safely, because the adjacent duodenum can only tolerate a maximum of 50 Gy without causing bleeding ulcers or wall perforation. Unfortunately for patients with unresectable pancreatic cancer, there are currently no effective treatments that specifically protect the GI tract from this radiotoxicity, and thus ablative radiotherapy in non-resectable pancreatic cancer is currently impractical.

In order to ensure the therapeutically effective accretion of the cytoprotectant API in the tissue adjacent to the radiated organ prior to the commencement of radiation, it is necessary to be able to ascertain the amount and location of the cytoprotectant API.

### SUMMARY

In an exemplary implementation, provided is a method of protecting a portion of the small intestine from radiation damage during radiation therapy in a subject in need thereof, using a nasal tube having a proximal end and a distal end, the method comprising: inserting the distal end of the nasal tube to a location in the portion of the small intestine adjacent to an organ or a tissue sought to be irradiated; validating the location of the distal end; administering to the portion of the small intestine a composition comprising a cytoprotectant pro-drug or a drug composition, wherein the cytoprotectant pro-drug or drug composition is configured to accrete in a wall of the portion of the small intestine adjacent to the irradiated organ or tissue; and exposing the organ or the tissue sought to be irradiated to ablative radiation.

In another exemplary implementation, provided herein is a method of use of a nasal tube having a proximal end and a distal end for the protection of at least one of: a duodenum, and a jejunum, each from radiation damage during ablative radiation, the method comprising: inserting the distal end of the nasal tube to a location in the portion of the small intestine adjacent to an organ or a tissue sought to be irradiated, wherein the distal end of the nasal tube is further coated with a radio-opaque composition; using an imaging module, validating the location of the distal end; through the nasal tube, administering to the portion of the small intestine an effective amount of a composition comprising: a cytoprotectant pro-drug or a drug composition, wherein the cytoprotectant pro-drug or drug composition is configured to accrete in a wall of the portion of the small intestine adjacent to the irradiated organ or tissue; and a bio-adhesive composition exposing the organ or the tissue sought to be irradiated to fractionated stereotactic body radiation, of between 3 and 5 fractions of 10 Gray (Gy) and 17 Gy per fraction, so long as the total radiation dose is above 50 Gy, over a predetermined number of sessions.

### BRIEF DESCRIPTION OF THE DRAWINGS

The features of the methods for the use of nasal tubes in targeted delivery of compositions of radioprotectants to a predetermined location adjacent an organ or a tissue sought to be irradiated, will become apparent from the following detailed description when read in conjunction with the drawings, which are exemplary, not limiting, and wherein like elements are numbered alike in several figures and in which:

FIG. 1, is a schematic of an exemplary implementation of the nasal tube

FIG. 2, is an image showing the location of the distal end of the nasal tube;

FIG. 3A, and FIG. 3B illustrate different implementations of the nasal tube distal tip:

FIG. 4, is a schematic illustrating the experimental design used; and

FIG. 5, depicts the duodenal strips.

While the disclosure is amenable to various modifications and alternative forms, specifics thereof have been shown by way of example in the figures and will be further described in detail hereinbelow. It should be understood, however, that the intention is not to limit the disclosure to the particular exemplary implementations described. On the contrary, the intention is to cover all modifications, equivalents, and alternatives.

### DETAILED DESCRIPTION

Provided herein are exemplary implementation of methods and uses of nasal tubes in targeted delivery of compositions of radioprotectants to a predetermined location adjacent an organ or a tissue sought to be irradiated in a subject in need thereof.

For example, achieving a cure for Pancreatic Cancer (PC) is currently limited to surgically resecting early-diagnosed disease, while patients with borderline resectable/potentially resectable (BRPC) or locally advanced pancreatic cancer (LAPC) face challenges, including tumor involvement in critical abdominal vessels making surgery difficult or impossible, contraindications due to aggressive features like elevated CA19-9, or other medical comorbidities.

Pancreatic cancer requires a biologically equivalent dose of more than 77 Gy to have a clinical (tumoricidal) benefit. Currently, this is not practicable for most pancreatic tumors unless they are in a location that is at least 1 cm away from the bowel wall. For example, external beam radiotherapy, which is typically delivered daily over 5-6 weeks using three-dimensional (3D) conformal or intensity-modulated radiation therapy (IMRT), and still remains the predominant treatment regimen, is characterized by its limited ability to spare bowel structures and the necessity for large treatment fields encompassing the pancreas and adjacent nodal areas, leading to elevated toxicity rates; furthermore, conventionally fractionated doses ranging from 40 to 60 Grays (Gy), which are derived from the tolerability of large-field radiation on the stomach and duodenum, have demonstrated minimal to negligible impact on overall patient survival.

In addition to the use of oral WR-2721 for improving the outcomes for pancreatic cancer patients, a similar strength Gy may be required for other abdominal or pelvic cancers that cannot be treated definitively with radiation due to GI toxicity, such as hepatobiliary tumors, retroperitoneal sarcomas, or metastatic disease within the abdomen.

Accordingly and in an exemplary implementation, provided herein is a method of protecting a portion of the small intestine from radiation damage during radiation therapy in a subject in need thereof, using a nasal tube having a proximal end and a distal end, the method comprising: inserting the distal end of the nasal tube to a location in the portion of the small intestine adjacent to an organ or a tissue sought to be irradiated; validating the location of the distal end; administering to the portion of the small intestine a composition comprising a cytoprotectant pro-drug or a drug composition, wherein the cytoprotectant pro-drug or drug composition is configured to accrete in a wall of the portion of the small intestine adjacent to the irradiated organ or tissue; and exposing the organ or the tissue sought to be irradiated to ablative radiation.

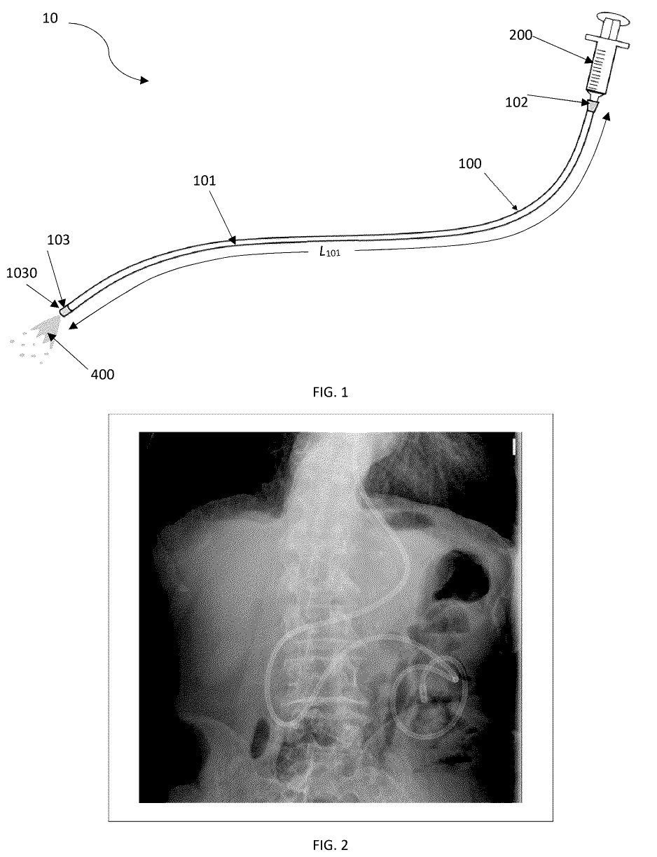

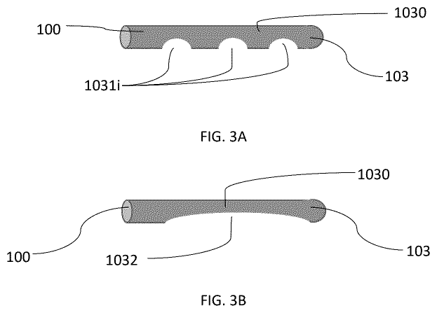

The nasal tube (see e.g., FIG. 1), can be a nasojejunal tube, a nasoduodanal tube or any other gastric tube sized, adapted and configured to extend beyond the stomach. As illustrated in FIG. 1, the nasal tube 100 is comprised of a body lumen 101 having proximal end 102 in liquid communication with a reservoir 200 containing the composition comprising the cytoprotectant pro-drug or the drug composition 400. The reservoir can be pressurized and be operable to deliver the cytoprotectant pro-drug or the drug composition at a predetermined rate, for example, between 2 milliliter (ml) per minute and 200 ml per minute. In an exemplary implementation, the nasal tube is made of flexible biocompatible polymers. These can be, for example: Polyurethane, Silicone, or Polyethylene, and the material selection can also be affected by the desired stiffness or flexibility of the tube, the duration of use, and subject-specific requirements (e.g., sensitivity to silicon). Furthermore, the distal end 103 of nasal tube 100 can be coated with a radiopaque coating 1030, used to enhance visibility during imaging procedures, a radiopaque coating can be applied to the distal tip of a nasoduodanal tube. The radiopaque coating 1030 contains a substance that is visible on X-ray or fluoroscopic images, allowing for better visualization and accurate placement confirmation. That substance can be for example, Barium Sulfate (BaSO₄), Bismuth Subcarbonate (Bi₂O₂(CO₃)), or Tungsten (W).

As illustrated in FIG. 3, the distal end 103 of the nasal tube 100 is operable to direct, or, in other words point the liquid composition comprising the cytoprotectant pro-drug or the drug composition to a predetermined location on the wall of the portion of the small intestine adjacent to the irradiated organ or tissue. For example, distal end 103 can have a plurality of perforations aligned ventrally 1031i with a fiducial designating their radial position close to the distal end of nasal tube 100, allowing the physician administering the composition to direct the liquid composition to the radial section of the wall that would provide the optimal protection. Similarly, distal end 103 can have a single aperture 1032 sized and configured to direct the liquid composition of the cytoprotectant pro-drug or the drug, allowing the physician administering the composition to direct the liquid composition to the radial section of the wall that would provide the optimal protection. Other configurations allowing pointing the liquid composition of the cytoprotectant pro-drug or the drug towards the radial portion of the small intestine (or other body lumen) are also contemplated.

In an exemplary implementation, the cytoprotectant pro-drug or the drug composition comprises at least one of: ergotamine, amifostine, Amifostine thiol-amifostine, 2-[(3-Aminopropyl)amino]ethanethiol dihydrochloride (hereinafter WR-1065) and pyridoxine.

In another exemplary implementation, the cytoprotectant pro-drug is the prodrug S-2-(3-aminopropylamino)ethyl dihydrogen phosphorothioate (hereinafter WR-2721) having the formula:

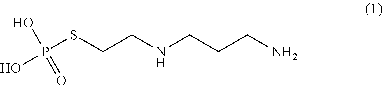 [SMILES: NCCCNCCSP(=O)(O)O]

given via the nasal tube before radiation, whereby, the pro-drug is rapidly activated by endogenous digestive enzymes in, for example, the duodenum and jejunum to its active 2-[(3-Aminopropyl)amino]ethanethiol dihydrochloride (hereinafter WR-1065). It is noted, that the pro-drug can also includes also its free mono-base or di-base conjugate, devoid of the respective HCl and any other pharmaceutically acceptable salt formation once passage into or through the duodenum, as well as metabolite having the formula:

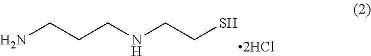

Due to the increased expression of non-tissue specific alkaline phosphatase in the intestine, activated form of WR-2721 would accumulate in high concentrations in the intestines and provide selective localized radioprotection with fewer systemic side effects. This can be useful during radiation for pancreatic cancer, since the duodenum and jejunum are dose-limiting organs preventing ablative treatments.

An “effective amount” of a subject compound, with respect to the pharmaceutical compositions, methods and uses, refers to an amount of the cytoprotective pro-drug in a preparation which, when applied as part of a desired dosage regimen (dose, formulation, frequency), prevents from bringing about, e.g., a negative change in rate of survival of a cell according to clinically acceptable standards.

In an exemplary implementation, the pro-drug is WR-2721. As used herein, the term “pro-drug” refers to a pharmacologically inactive form of a compound that undergoes biotransformation prior to exhibiting its pharmacological effect(s). A pro-drug is one that is converted in vivo by a subject after administration into a pharmacologically active form of the compound in order to produce the desired pharmacological effect. After administration to the subject, the pharmacologically inactive form of the compound is converted in vivo under the influence of biological fluids and/or enzymes into a pharmacologically active form of the compound. Although metabolism occurs for many compounds primarily in the liver and/or kidney, almost all other tissues and organs, especially the lung, are able to carry out varying degrees of metabolism. Pro-drug forms of compounds can be utilized, for example, to improve bioavailability, mask unpleasant characteristics such as bitter taste, alter solubility for intravenous use, or to provide site-specific delivery of the compound. Reference to a compound herein includes pro-drug forms of a compound and the drug conjugate (active form).

The dosage forms of WR-2721 can be also be a part of a composition comprising salt of a chelating agent selected from the group consisting of EDTA, EGTA, citrate and therapeutically acceptable salts thereof. A preferred formulation can be made with the pharmacologically required dose of WR-2721 being between about 50 mg/unit of dosage form and about 2000 mg/unit dosage form or NMT 2000 mg/dosage form unit for example, between about 125 mg/and about 750 mg or about 250 mg.

In several tissue/organ imaging technologies (e.g., X-ray and computed tomography (CT), positron emission tomography (PET) and single photon emission computed tomography (SPECT), magnetic resonance imaging (MRI), ultrasound imaging, optical imaging, collectively—imaging module) the contrast in the generated image used to validate the location of the distal end of the nasal tube, may be enhanced by coating the distal end with a “contrast agent”. For example, in MRI, the contrast agent is operable to affect the spin re-equilibration (e.g., time) characteristics of nuclei (the “imaging nuclei” which generally are protons and more especially water protons) which are responsible for the resonance signals from which the images are generated.

The enhanced contrast thus obtained enables the physician to be more clearly visualize the distal end of the nasal tube by increasing or by decreasing the brightness of the image of the particular organ or tissue relative to that of the distal end of the nasal tube. MRI employs a magnetic field, radio frequency energy and magnetic field gradients to make images of the body. The contrast or signal intensity differences between tissues mainly reflect the T1 (longitudinal) and T2 (transverse) relaxation values and the proton density (effectively, the free water content) of the tissues. In changing the signal intensity in a region of a patient by the use of a contrast agent, several possible approaches are available. For example, a contrast medium can be used to change either the T1, the T2 or the proton density of the tissue containing the contrast agent. As used herein the term “contrast” refers to the relative difference of signal intensities in two adjacent regions of an image. Image contrast is heavily dependent on the chosen imaging technique (i.e., TE, TR, TI), and is associated with such parameters as proton density and T1 or T2 relaxation times. Accordingly, the radio-opaque contrast tracer used to coat the distal end of the nasal tube in the methods and uses disclosed herein, can be, for example, zirconium oxide, aluminum oxide, barium sulphate, sodium amidotrizoate, meglumine amidotrizoate, sodium diatrizoate, sodium calcium edetate, Iodixanol, or triphenyl bismuth, diatrizoate (see e.g., FIG. 3), metrizoate, iothalamate, ioxaglate, iopamidol, iohexol, ioxilan, iopromide, iodixanol, iobitridol, ioversol, or a composition comprising one or more of the foregoing.

In the context of the disclosure, the term “radio-opaque agent” refers to any substance or agent which blocks, absorbs, scatters, or reflects any radiation outside the visible light spectrum, including, but not limited to, X-rays (in the wavelength range of 0.01 to 10 nm), beta rays (e.g., having velocities of about 35,000 to 180,000 miles per second), gamma rays (having an energy in the range of 10⁴ to 10⁷ eV), radiation used in radiation therapy (e.g., therapy to treat cancer), and other harmful radiation (such as that resulting from nuclear disasters and nuclear weapons). Suitable radio-opaque agents include, but are not limited to, those comprising platinum, gold, silver, bismuth, mercury, lead, barium, calcium, zinc, aluminum, iron, gallium, iodine, tungsten, and any combination of any of the foregoing. Other suitable radio-opaque agents include, but are not limited to, those commercially available as radio-opaque agents for medical uses, such as ionic and nonionic intravenous radiocontrast agents, diagnostic barium and gastrographin preparations, and gallium preparations.

Furthermore, in certain exemplary implementations, the cytoprotectant pro-drug or the drug composition can further comprise a bio-adhesive composition (interchangeable with ‘mucoadhesive composition), which is adapted to form the bio-adhesive upon mixing with a body fluid, the bio-adhesive configured to adhere the cytoprotectant pro-drug or the drug composition to a radial portion of the wall of a body lumen, such as the duodenum and jejunum. The bio-adhesive is, in an exemplary implementation, a mucoadhesive polymer composition, configured to prolong the residence time of the dosage form at the site of absorption (e.g., the duodenum, or jejunum), following the calculated lag in release, and to facilitate intimate contact of the dosage form with the underlying duodenum 1 inside surface to improve and enhance the efficacy of the therapeutically effective amount of the API. In the context of the disclosure, the term “bio-adhesive”, or “mucoadhesive” denotes a compound exhibiting an affinity for a mucosal surface. Mucoadhesive polymers are typically polymers having hydrogen bonding groups. See e.g. http://en.wikipedia.org/wiki/Bioadhesive#Mucoadhesion. “Mucoadhesion is the ability of materials to adhere to mucosal membranes in the human body and provide a temporary retention”. “Excellent mucoadhesive properties are typical for hydrophilic polymers possessing charged groups and/or non-ionic functional groups capable of forming hydrogen bonds with mucosal surfaces.” [Macromol Biosci. 2011 Jim 14; 1J (6): 748-64. doi: 10.1002/mabi.201000388.Epub 2010 Dec. 27]. In an exemplary implementation, the bio-adhesive composition is a mucoadhesive composition that is comprised of hydroxylpropyl cellulose (HPC), hydroxypropylmethylcellulose (HPMC), Hypromellose, starch, polyvinylpyrollidone (PVP), xanthan gum, thiolated chitosan, or a composition comprising one or more of the foregoing. In an exemplary implementation, the bio-adhesive compositions comprises between about NLT 2% (w/w tablet).

In certain exemplary implementations, the bioadhesive composition comprises Povidone (also known as polyvinylpyrrolidone or PVP). Povidone is a water-soluble polymer that exhibits mucoadhesive properties and can be configured to enhance the residence time of pharmaceutical compositions on mucosal surfaces of the small intestine, particularly the duodenum. The Povidone may have various molecular weights, with Povidone K25 (having a weight average molecular weight of approximately 30,000 Daltons) being suitable for formulations intended for delivery via nasal tubes to the duodenum. For example, the cytoprotectant composition delivered using the nasal tube disclosed, can comprise Povidone having a concentration ranging from between about 2% and about 15% (w/v), or between about 5% and about 10% (w/v), or specifically at about 5% (w/v), or about 10% (w/v). These concentrations of Povidone can provide sufficient mucoadhesive properties to prolong contact time with the duodenal mucosa while maintaining suitable viscosity, characteristics for delivery through a nasal tube. The Povidone-containing formulation is adapted to exhibit enhanced tissue permeability compared to formulations without Povidone, resulting in improved accumulation of the cytoprotectant in the target tissue.

Furthermore, in the resulting formulation Povidone K25 used at a concentration of 10% (w/v) in an aqueous solution containing Amifostine at 150 mg/mL, demonstrates, significantly enhanced tissue penetration and accumulation in duodenal tissue. In an exemplary implementation, such formulations achieve tissue concentrations of approximately 14 to 15 micromolar per gram of tissue (μM/g) within 15 to 30 minutes of application to the duodenal mucosa, representing an improvement of approximately 1.5-fold to 2-fold compared to formulations without Povidone.

In an exemplary implementation, the cytoprotectant pro-drug or the drug composition has a viscosity of between about 25 centipoise (cP) and about 1500 cP, while in flow in the nasal tube, and be adapted to undergo in-situ gelation. In-situ gelation refers to the process of a liquid composition transforming into a gel-like state in response to certain triggers or stimuli. When considering in-situ gelation in the context of the small intestine, there are several triggers that can induce gel formation. These triggers can be, for example: pH, Temperature, Ion concentration, enzymatic activity, or their combination.

In certain other implementations, the cytoprotectant agent, such as ergotamine, amifostine, Amifostine thiol-amifostine, 2-[(3-Aminopropyl)amino]ethanethiol dihydrochloride (hereinafter WR-1065) or pyridoxine, are embedded, encapsulated or entrapped in a hydrogel configured to release the embedded, encapsulated or entrapped API in response to change in pH in transitioning from the stomach to the duodenum or jejunum, for example in transitioning from a pH<4.7 to a pH>5.2. These hydrogels can be formed from, for example polycaprolactone methacrylic acid graft copolymer (MAC-g-PCL).

For example, Polycarbophil (also known as calcium polycarbophil) is a pH-sensitive biopolymer that can undergo gelation in response to the higher pH environment of the small intestine, the duodenum, or jejunum. Alternatively, Poloxamer 407 (Pluronic® F127), a thermosensitive biopolymer can undergo gelation when the temperature reaches a specific range. It forms a gel at body temperature, making it suitable for in-situ gelation in the small intestine. Additionally, or alternatively Sodium alginate is an ion-sensitive biopolymer that can gel in the presence of calcium ions. When a composition containing sodium alginate encounters an increase in calcium ion concentration in the small intestine, it can undergo gelation, The introduction of calcium ions can be done using a double lumen nasal tube. In addition, Chitosan is a biopolymer that can undergo enzymatic degradation by the enzyme lysozyme, which is present in the small intestine. By incorporating chitosan into the composition, the enzymatic activity in the small intestine can trigger gelation.

In yet another exemplary implementation, the cytoprotectant composition comprises Amifostine (WR-2721) at a concentration of between about 100 mg/mL and about 200 mg/mL, or about 150 mg/mL, in combination with Povidone K25 at a concentration of between about 5% and about 10% (w/v), for example, about 10% (w/v), in an aqueous solution. This formulation is adapted to provide both adequate dosing of the cytoprotectant pro-drug and enhanced mucoadhesion thereby prolonging residence time in the duodenum, and consequently maximizing tissue uptake and accumulation of both the pro-drug (WR-2721) and its active metabolite (WR-1065) in the duodenal tissue prior to radiation therapy. The Povidone K25 in this formulation can have a molecular weight of approximately 30,000 Daltons and exhibit pH-independent mucoadhesive properties across the physiological pH range of the duodenum (e.g, pH 5.5 to 7.5).

In the context of the disclosure, the term “pharmaceutically acceptable salt” refers to salts prepared from pharmaceutically acceptable nontoxic acids and bases, including inorganic and organic acids and bases. The term “pharmaceutically acceptable salt” also refers to a salt prepared from an active pharmaceutical ingredient (API), referring to the cytoprotectant pro-drug or the drug in the composition having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. Moreover, the term “pharmaceutically acceptable salt” also refers to a salt prepared from the API, e.g., amifostine, 2-[(3-Aminopropyl)amino]ethanethiol dihydrochloride (hereinafter WR-1065), having a basic functional group, such as an amino functional group, and a pharmaceutically acceptable inorganic or organic acid. Suitable acids can be, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

In the context of the disclosure, the language “at least one of: a first tissue, and a first organ” (in other words, first tissue and/or organ) is intended to describe the tissue and/or organ where the tumor sought to be irradiated is located. Conversely, the language “at least one of: an adjacent second organ, and an adjacent second tissue” is intended to describe the tissue and/or organ that are prohibitively sensitive to irradiation and are adjacent to the tumor location. For example, the first organ would be the pancreas head, and the second tissue would be the duodenum wall. Similarly, the first organ can be the prostate in the second organ can be the GI tract.

In an exemplary implementation, the tissues sensitive to radiation is the duodenum and/or jejunum. For example, given as conventionally fractionated therapy, typical limits for maximum radiation dose to the duodenum are thought to be about 50 Gray (Gy) to one-third of the organ or 40 Gy to the entire organ, with recent guidelines recommending that only 195 cm³ of small bowel receive >45 Gy. Conversely, as disclosed herein, biologically effective doses in (large) excess of 55 Gy may be necessary to achieve a high probability of tumor control.

As indicated, the step of exposing the organ or the tissue sought to be irradiated to ablative radiation comprises: using fractionated stereotactic body radiation therapy, exposing the predetermined location on the organ or the tissue sought to be irradiated, to between 1 and about 5 irradiation fractions. Accordingly and in an exemplary implementation, the step of exposing the organ or the tissue sought to be irradiated to ablative radiation to a therapeutically effective radiation dose comprises using stereotactic body radiation therapy (SBRT), administrating to the patient a total radiation dose of between about 10 Gy, and about 17 Gy per fraction for a total of between one and five fractions (50-85 Gy), which would be a total BED10 of 50 Gy-208 Gy in three to five fractions or an D2EQ of 50 Gy to 173.3 Gy on three to five fractions. It is noted that the minimum radiation exposure will always be above 50 Gy.

$$D 2 equivalent ( D 2 EQ ) = D 1 ( α / β + d 1 ) / ( α / β + d 2 ) \qquad ( Equ . 1 )$$

where D2=equivalent total dose, D1=initial total dose, d1=initial dose/fraction and d2=wanted dose/fraction

In stereotactic body radiation therapy (SBRT), a single or limited number of focused, high dose radiation fractions are configured to be delivered to the tumor, which enables the delivery of ablative doses to the tumor and immediately adjacent tissues. In an exemplary implementation, SBRT can be an alternative to resection when a critical structure, which precludes its surgical resection, is presented. Moreover, in another exemplary implementation, the methods disclosed further comprise treatment planning using, for example, respiratory-correlated cone-beam computed tomography (4D-CT), with abdominal compression to limit the respiratory-associated movement of tumor during the step of delivering the fractionated radiation. In certain exemplary implementations, fiducial markers are used during the course of treatment to actively track tumor movement.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the tumor(s) includes one or more tumor). Reference throughout the specification to “one exemplary implementation”, “another exemplary implementation”, “an exemplary implementation”, and so forth, means that a particular element (e.g., feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In implementations utilizing Povidone-based formulations, the viscosity of the composition can be tailored e.g., by adjusting the concentration of Povidone. For example, formulations containing Povidone K25 at concentrations of 5% (w/v) exhibit viscosity in the range of between about 25 cP, and about 100 cP at room temperature, while formulations containing Povidone K25 at about 10% (w/v) exhibit viscosity in the range of between about 50 cP and about 200 cP at room temperature. These viscosity ranges can be adapted to ensure that the formulation can be readily administered through the nasal tube, while simultaneously providing sufficient mucoadhesion upon contact with the duodenal mucosa. The relatively low viscosity during flow through the nasal tube can be beneficial, as it prevents clogging of the tube, while the mucoadhesive properties of Povidone ensure prolonged contact with the target tissue after delivery.

Alternatively or additionally, the mucoadhesive composition may comprise polymers that exhibit pH-independent mucoadhesion, such as Povidone (polyvinylpyrrolidone, PVP). Unlike pH-responsive polymers such as Polycarbophil that require specific pH conditions for gelation, Povidone-based formulations can be adapted to maintain their mucoadhesive properties across a broad pH range, making them particularly suitable for delivery to regions of the gastrointestinal tract where pH may vary, and/or be difficult to predict with precision. The pH-independent nature of Povidone mucoadhesion ensures consistent performance regardless of individual patient variations in gastric acid production, buffering capacity, or co-administration of acid-suppressing medications.

### EXAMPLES

### Example I: Preparation of Povidone-Based Amifostine Formulations

The following examples are provided to illustrate certain embodiments of the disclosure and are not intended to limit the scope of the claims. Those skilled in the art will recognize that various modifications can be made without departing from the spirit and scope of the disclosure.

Test Formulation (AMF-PVP-080725): An oral solution of Amifostine in Povidone was prepared as follows. Povidone K25 (polyvinylpyrrolidone having an average molecular weight of approximately 30,000 Daltons) was dissolved in purified water at a concentration of 10% (w/v) by stirring overnight at room temperature. Amifostine trihydrate (batch 04221001 from Lianyuangang Runzhong Pharmaceutical Co., LTD) was added to the Povidone solution to achieve a final Amifostine concentration of 150 mg/mL. The mixture was stirred for approximately 2 hours at room temperature until complete dissolution was achieved. The resulting formulation was a clear to slightly opalescent, low-viscosity aqueous solution suitable for administration via a nasal-duodenal tube.

Reference Formulation: A reference formulation consisting of Amifostine at 150 mg/mL in 0.9% saline (normal saline, pH approximately 5.5 to 6.5) was prepared by dissolving Amifostine trihydrate in physiological saline solution with stirring at room temperature for approximately 1 to 2 hours.

Viscosity Measurements: The viscosity of the Test Formulation (10% Povidone K25 with 150 mg/mL Amifostine) was measured at room temperature and found to be in the range of 75 to 150 centipoise, suitable for flow through nasal tubes with internal diameters of 2 to 4 mm. The Reference Formulation exhibited viscosity similar to water (approximately 1 to 2 centipoise). Additional formulations containing Povidone K25 at 5% (w/v) with Amifostine at concentrations ranging from 100 mg/mL to 200 mg/mL were similarly prepared and exhibited viscosities in the range of 30 to 80 centipoise.

### Example II: Ex Vivo Tissue Permeation Study Design

Objective: Evaluate and compare the permeation of Amifostine (AMF, WR-2721) and its active metabolite Amifostine thiol (AMFT, WR-1065) into duodenal tissue following application of the Test Formulation (AMF-PVP, containing 10% Povidone K25) versus the Reference Formulation (AMF-Saline) using an ex vivo porcine duodenal tissue model.

Tissue Preparation: Fresh porcine small intestine was obtained from a local abattoir within 2 hours of sacrifice. The duodenal segment (identified as the first 30 to 40 cm of small intestine distal to the pylorus) was isolated, and the tissue was carefully cleaned with ice-cold phosphate-buffered saline (PBS, pH 7.4) to remove luminal contents and debris. The tissue was kept on ice throughout the preparation process. Strips of full-thickness duodenal tissue measuring approximately 4 cm in length and 2 cm in width were prepared using surgical scissors. Each tissue strip was visually inspected to ensure integrity of the mucosal surface, and any damaged tissues were discarded.

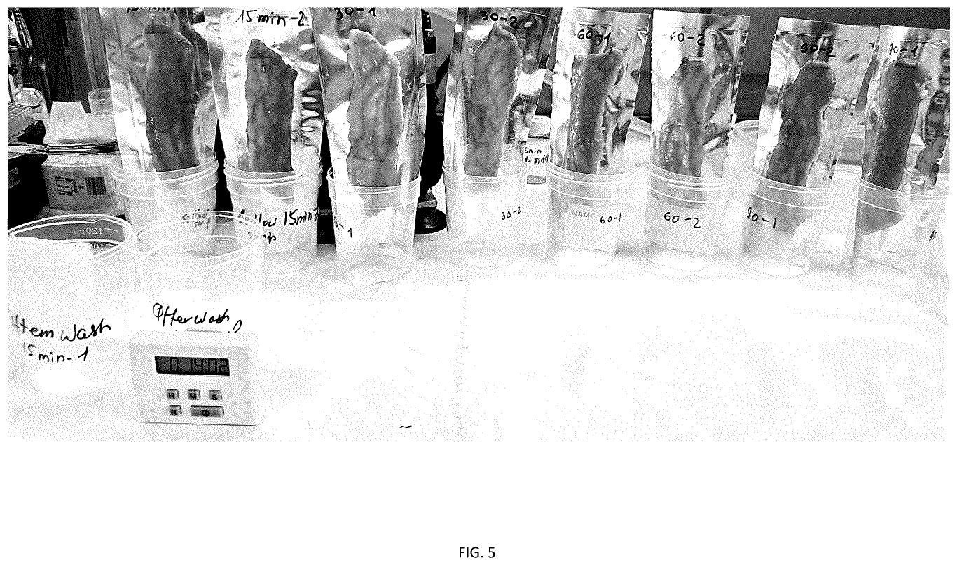

Experimental Setup: The ex vivo permeation experiments utilized a vertical tissue mounting apparatus. Each tissue strip was mounted vertically with the mucosal (luminal) surface facing upward (see e.g., FIG. 5). A volume of 2 mL of either Test Formulation (AMF-PVP) or Reference Formulation (AMF-Saline) was carefully pipetted onto the mucosal surface of each tissue strip to form a thin layer covering the entire surface area. The tissue strips with applied formulation were maintained at 37° C. in a humidified environment to simulate physiological conditions.

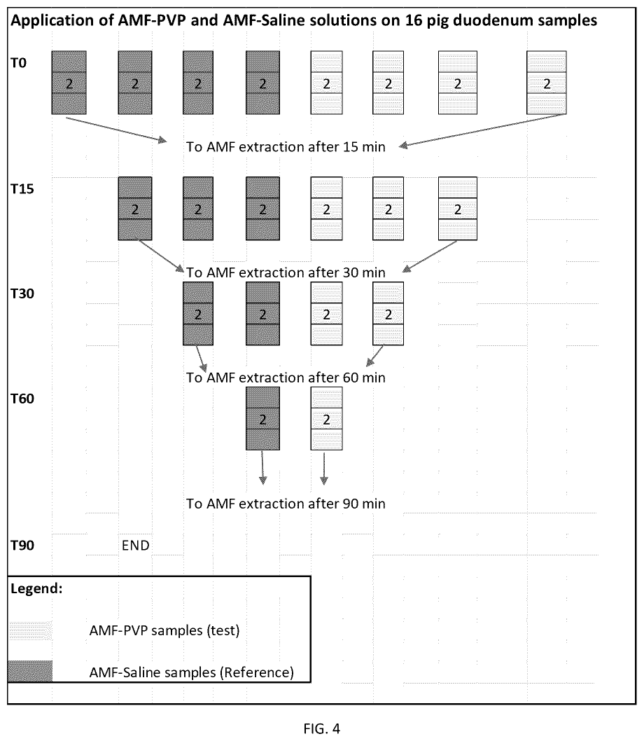

Eight tissues were tested for each formulation (see e.g., FIG. 4); 2 mL of the formulation was applied; The residual solution was collected at each period and analyzed for AMF and AMFT; Two tissues were washed at each period with 5 mL of acetate buffer pH 5.5; The wash solution was collected and analyzed for AMF and AMFT; Each tissue was cut into 3 pieces (top, mid, bottom); 2 mL of phosphate buffer, pH 6.5, was added to the AMF-Saline sample, and 3 mL to the AMF-PVP sample; The samples were then homogenized and centrifuged; 200 μL of TFA-ACN solution was added to 400 μL of supernatant, and samples were centrifuged again; and 5 μL were injected into the HPLC for AMF and AMF thiol analysis

Sampling and Time Points: Duplicate tissue samples were collected at each of the following time points after formulation application: 15 minutes, 30 minutes, 60 minutes, and 90 minutes. At each time point, the tissue strip was removed from the mounting apparatus, and any excess formulation on the mucosal surface was carefully collected and quantified. The tissue strip was then briefly rinsed with 5 mL of PBS to remove residual surface formulation, and the wash solution was collected separately. Each tissue strip was then divided into three segments (top, middle, and bottom) to assess uniformity of drug penetration along the length of the tissue. Each tissue segment was weighed and immediately frozen at −80° C. until analysis.

Analytical Methods: Tissue samples were thawed and homogenized in 5 mL of extraction buffer (0.1 M hydrochloric acid containing 10 mM EDTA as a stabilizer to prevent oxidation of the thiol groups). The homogenate was centrifuged at 10,000×g for 10 minutes at 4° C., and the supernatant was filtered through a 0.22 μm PVDF syringe filter. The concentrations of Amifostine (WR-2721) and Amifostine thiol (WR-1065) in the tissue extracts, excess formulation, and wash solutions were determined by high-performance liquid chromatography with photodiode array detection (HPLC-PDA) using a validated analytical method. The HPLC method employed a C18 reversed-phase column with gradient elution using aqueous phosphate buffer and acetonitrile as mobile phases. Detection was performed at 214 nm for Amifostine and 232 nm for Amifostine thiol. Standard curves were prepared over the concentration range of 0.1 to 100 μg/mL with correlation coefficients (R²) exceeding 0.999.

Mass Balance Calculations: For each sample, a mass balance was calculated to account for the total amount of Amifostine applied to the tissue. The mass balance included: (1) Amifostine and Amifostine thiol in the excess formulation remaining on the tissue surface; (2) Amifostine and Amifostine thiol in the wash solution; and (3) Amifostine and Amifostine thiol extracted from the tissue segments (top, middle, and bottom). Recovery of applied Amifostine ranged from 84% to 103%, with most samples exhibiting recovery between 90% and 100%, demonstrating adequate mass balance closure and reliability of the analytical methods.

### Example III: Quantitative Results of Tissue Permeation Study

Tissue Concentration Data: The amounts of Amifostine (AMF) and Amifostine thiol (AMFT) in duodenal tissue following application of the Test Formulation (AMF-PVP, 10% Povidone K25) and Reference Formulation (AMF-Saline) at different time points are summarized below:

### Test Formulation (AMF-PVP)—15-Minute Time Point

Two replicate tissue samples (AMF-P tissue 15-1 and AMF-P tissue 15-2) were analyzed. The combined tissue weights were 3.64 grams and 3.75 grams, respectively. The total amounts of AMF and AMFT extracted from the three tissue segments (top, middle, bottom) were 11.40 mg and 11.17 mg (average: 11.29 mg) for the two replicates. This corresponded to tissue concentrations of 3.10 mg/g and 2.98 mg/g (average: 3.04 mg/g) or, expressed in molar terms, approximately 14.2 μM/g and 13.9 μM/g (average: 14.05 μM/g) based on the molecular weight of Amifostine of 214 g/mol.

### Test Formulation (AMF-PVP)—30-Minute Time Point

The combined tissue weights for two replicates were 2.99 grams and 3.15 grams. The total amounts of AMF and AMFT in tissue were 9.08 mg and 7.52 mg (average: 8.30 mg), corresponding to tissue concentrations of 3.04 mg/g and 2.39 mg/g (average: 2.71 mg/g) or approximately 14.2 μM/g and 11.2 μM/g (average: 12.7 μM/g).

### Test Formulation (AMF-PVP)—60-Minute Time Point

The combined tissue weights for two replicates were 4.87 grams and 4.58 grams. The total amounts of AMF and AMFT in tissue were 17.97 mg and 9.54 mg (average: 13.76 mg), corresponding to tissue concentrations of 3.69 mg/g and 2.08 mg/g (average: 2.89 mg/g) or approximately 17.2 μM/g and 9.7 μM/g (average: 13.5 μM/g).

### Test Formulation (AMF-PVP)—90-Minute Time Point

The combined tissue weights for two replicates were 1.94 grams and 2.09 grams. The total amounts of AMF and AMFT in tissue were 5.61 mg and 6.85 mg (average: 6.23 mg), corresponding to tissue concentrations of 2.90 mg/g and 3.28 mg/g (average: 3.09 mg/g) or approximately 13.5 μM/g and 15.3 μM/g (average: 14.4 μM/g).

### Reference Formulation (AMF-Saline)—15-Minute Time Point

The combined tissue weights for two replicates were 3.47 grams and 4.15 grams. The total amounts of AMF and AMFT in tissue were 5.49 mg and 6.00 mg (average: 5.75 mg), corresponding to tissue concentrations of 1.58 mg/g and 1.45 mg/g (average: 1.51 mg/g) or approximately 7.4 μM/g and 6.8 μM/g (average: 7.1 μM/g).

### Reference Formulation (AMF-Saline)—30-Minute Time Point

The combined tissue weights for two replicates were 4.91 grams and 3.16 grams. The total amounts of AMF and AMFT in tissue were 7.98 mg and 6.84 mg (average: 7.41 mg), corresponding to tissue concentrations of 1.63 mg/g and 2.16 mg/g (average: 1.89 mg/g) or approximately 7.6 μM/g and 10.1 μM/g (average: 8.85 μM/g).

### Reference Formulation (AMF-Saline)—60-Minute Time Point

The combined tissue weights for two replicates were 4.74 grams and 4.86 grams. The total amounts of AMF and AMFT in tissue were 8.41 mg and 7.58 mg (average: 8.00 mg), corresponding to tissue concentrations of 1.77 mg/g and 1.56 mg/g (average: 1.67 mg/g) or approximately 8.3 μM/g and 7.3 μM/g (average: 7.8 μM/g).

### Comparative Analysis Across all Time Points

When averaged across all time points (15, 30, 60, and 90 minutes), the mean tissue concentration of total Amifostine species (AMF+AMFT) for the Test Formulation (AMF-PVP) was 11.76 mg per tissue sample with an average tissue weight of 3.74 grams, yielding a mean concentration of 3.14 mg/g or 14.7 μM/g. In comparison, the mean tissue concentration for the Reference Formulation (AMF-Saline) across the 15, 30, and 60-minute time points was 7.20 mg per tissue sample with an average tissue weight of 3.69 grams, yielding a mean concentration of 1.95 mg/g or 9.1 μM/g. This represents a 1.61-fold improvement in tissue penetration and accumulation for the Povidone-containing formulation compared to the saline formulation. Alternatively stated, the Test Formulation achieved approximately 61% greater tissue concentration of cytoprotectant compared to the Reference Formulation.

### Results:

The difference in tissue concentrations between the Test Formulation (AMF-PVP) and Reference Formulation (AMF-Saline) was statistically significant (p<0.05 by Student's t-test), indicating that the inclusion of 10% Povidone K25 in the formulation provides a reproducible and meaningful enhancement of Amifostine tissue penetration and accumulation in duodenal tissue.

### Example IV: Interpretation and Clinical Significance of Enhanced Tissue Permeation

The ex vivo tissue permeation studies described in Examples 2 and 3 demonstrate that formulation of Amifostine with Povidone K25 at a concentration of 10% (w/v) significantly enhances the penetration and accumulation of the cytoprotectant pro-drug in duodenal tissue. The observed 1.61-fold increase in tissue concentration (14.7 μM/g versus 9.1 μM/g) has important clinical implications for the practice of radioprotection during ablative radiation therapy for pancreatic cancer and other abdominal malignancies.

### Mechanism of Enhanced Permeation:

Without being bound by theory, the enhanced tissue permeation observed with the Povidone-containing formulation is believed to result from multiple factors. First, Povidone exhibits mucoadhesive properties that increase the residence time of the formulation on the duodenal mucosal surface, thereby extending the duration of contact between the drug and the tissue. This prolonged contact time increases the opportunity for drug molecules to diffuse across the mucosal epithelium and penetrate into deeper tissue layers. Second, Povidone may act as a permeation enhancer by transiently increasing the permeability of the mucosal barrier through interactions with epithelial cell membranes and tight junctions. Third, the presence of Povidone may create a favorable microenvironment at the tissue-formulation interface that promotes drug dissolution and maintains drug in a form that is readily available for absorption.

### Relevance to Radioprotection Efficacy:

Previous studies have demonstrated that the efficacy of oral Amifostine in protecting intestinal tissues from radiation damage is directly correlated with the concentration of the active cytoprotectant (WR-1065) that accumulates in the tissue prior to irradiation. Specifically, tissue concentrations of WR-1065 in the range of 10 to 20 μM/g have been shown to provide significant radioprotection in preclinical models. The mean tissue concentration of 14.7 M/g achieved with the AMF-PVP formulation falls within this efficacious range, whereas the lower tissue concentration of 9.1 μM/g achieved with the saline formulation approaches the lower threshold of efficacy. Thus, the Povidone-enhanced formulation is expected to provide superior radioprotection compared to formulations lacking Povidone, potentially enabling the delivery of higher ablative radiation doses to tumors while maintaining acceptable toxicity to normal duodenal tissue.

### Time Course of Tissue Accumulation:

The data presented in Example 3 demonstrate that significant tissue accumulation of Amifostine occurs as early as 15 minutes after formulation application, with tissue concentrations in the range of 7 to 14 μM/g being achieved within this short timeframe. This rapid onset of tissue accumulation is clinically advantageous, as it allows for a relatively short interval between drug administration and commencement of radiation therapy. In a typical clinical protocol, the AMF-PVP formulation would be delivered to the duodenum via a nasal-duodenal tube approximately 15 to 30 minutes prior to the start of radiation treatment, allowing sufficient time for tissue accumulation of the cytoprotectant while minimizing the overall duration of the treatment session.

### Uniformity of Tissue Distribution:

Analysis of the individual tissue segments (top, middle, and bottom portions of each tissue strip) revealed that Amifostine and its active metabolite were distributed relatively uniformly along the length of the exposed tissue, with no consistent gradient or preferential accumulation in any particular region. This finding indicates that the AMF-PVP formulation, when applied to the duodenal mucosal surface, spreads uniformly and provides consistent drug delivery across the treated area. Such uniform distribution is desirable for radioprotection applications, as it ensures that all portions of the duodenum that may be within the radiation field receive adequate cytoprotectant coverage.

### Example V: Formulations with Povidone K25 at 5% (w/v)

Additional formulations were prepared containing Povidone K25 at a concentration of 5% (w/v) in combination with Amifostine at concentrations of 100 mg/mL, 150 mg/mL, and 200 mg/mL. These formulations exhibited lower viscosity compared to the 10% Povidone formulations (viscosity range of approximately 30 to 80 centipoise versus 75 to 150 centipoise), making them even more readily flowable through nasal tubes.

Preliminary ex vivo tissue permeation studies using the 5% Povidone K25 formulations demonstrated tissue concentrations of Amifostine in the range of 10 to 13 μM/g at the 15 to 30-minute time points, which was intermediate between the results obtained with the 10% Povidone formulation (approximately 14.7 μM/g) and the saline formulation (approximately 9.1 μM/g). These results indicate that Povidone concentrations in the range of 5% to 10% (w/v) provide a beneficial enhancement of tissue permeation, with higher Povidone concentrations generally providing greater enhancement.

The selection of Povidone concentration (5% versus 10%) for a particular clinical application may be optimized based on factors such as the desired viscosity for ease of administration, the target tissue concentration to be achieved, and the available timeframe between drug administration and radiation treatment. For applications where maximum tissue concentration is desired and viscosity is not a limiting factor, the 10% Povidone formulation may be preferred. For applications where ease of administration through smaller-diameter nasal tubes is a priority, the 5% Povidone formulation may be advantageous while still providing meaningful enhancement over non-Povidone formulations.

Accordingly and in an exemplary implementation, provided herein is a method of protecting a portion of the small intestine from radiation damage during radiation therapy in a subject in need thereof, using a nasal tube having a proximal end and a distal end, the method comprising: inserting the distal end of the nasal tube to a location in the portion of the small intestine adjacent to an organ or a tissue sought to be irradiated; validating the location of the distal end; administering to the portion of the small intestine an effective amount of a composition comprising a cytoprotectant pro-drug or a drug composition, wherein the cytoprotectant pro-drug or drug composition is configured to accrete in a wall of the portion of the small intestine adjacent to the irradiated organ or tissue; and exposing the organ or the tissue sought to be irradiated to ablative radiation, wherein (i) the nasal tube is a nasoduodanal tube, or nasojejunal tube, wherein (ii) the distal end of the nasal tube is operable to direct the composition comprising the cytoprotectant pro-drug or the drug composition to a predetermined location on the wall of the portion of the small intestine adjacent to the irradiated organ or tissue, (iii) the cytoprotectant pro-drug or the drug composition comprises at least one of: ergotamine, amifostine, Amifostine thiol-amifostine, 2-[(3-Aminopropyl)amino]ethanethiol dihydrochloride (hereinafter WR-1065) and pyridoxine, (iv) the cytoprotectant pro-drug or a drug composition comprises S-2-(3-amino propylamino)ethyl dihydrogen phosphorothioate, its active 2-[(3-Aminopropyl)amino]ethanethiol dihydrochloride (WR-1065), or their pharmaceutically accepted salt, wherein (v) the distal end of the nasal tube is further coated with a radio-opaque composition and the step of validating the location of the distal end further comprises imaging the expected location, the method (vi) further comprising adjusting the location of the distal end prior to administering the composition comprising the cytoprotectant pro-drug or the drug composition, wherein (vii) the composition comprising the cytoprotectant pro-drug further comprises a bio-adhesive composition, (viii) the bio-adhesive composition is comprised of hydroxylpropyl cellulose (HPC), hydroxypropylmethylcellulose (HPMC), Hypromellose, starch, polyvinylpyrollidone (PVP), xanthan gum, or a composition comprising one or more of the foregoing, wherein (ix) the cytoprotectant pro-drug or the drug composition has a viscosity of between about 25 centipoise (cP) and about 1500 cP, wherein (x) the organ or tissue sought to be protected is a duodenum, a jejunum, a large intestine, a rectum, an esophagus, or a small intestine, (xi) a pancreas, a uterus, a prostate, or a bladder, wherein (xii) the step of exposing the organ or the tissue sought to be irradiated to ablative radiation comprises: using fractionated stereotactic body radiation therapy, exposing the organ or the tissue sought to be irradiated to between 1 and about 5 irradiation fractions, (xiii) the fractionated stereotactic body radiation therapy is configured to expose the organ or the tissue sought to be irradiated to a total radiation dose of between about 50 Gy and about 208 Gy, wherein (xiv) the radiation is administered in between 3 and 5 fractions of 10 Gy and 17 Gy per fraction, so long as the total radiation dose is above 50 Gy, wherein (xv) the composition comprising the cytoprotectant pro-drug or the drug composition is configured to gel in-situ, and wherein (xvi) the in-situ gelation is triggered by pH change, temperature change, ionic concentration change, enzymatic activity or a combination thereof.

In another exemplary implementation, provided herein is a method of use of a nasal tube having a proximal end and a distal end for the protection of at least one of: a duodenum, and a jejunum, each from radiation damage during ablative radiation, the method comprising: inserting the distal end of the nasal tube to a location in the portion of the small intestine adjacent to an organ or a tissue sought to be irradiated, wherein the distal end of the nasal tube is further coated with a radio-opaque composition; using an imaging module, validating the location of the distal end; through the nasal tube, administering to the portion of the small intestine an effective amount of a composition comprising: a cytoprotectant pro-drug or a drug composition, wherein the cytoprotectant pro-drug or drug composition is configured to accrete in a wall of the portion of the small intestine adjacent to the irradiated organ or tissue; and a bio-adhesive composition exposing the organ or the tissue sought to be irradiated to fractionated stereotactic body radiation (SBRT), of between 3 and 5 fractions of 10 Gy and 17 Gy per fraction, so long as the total radiation dose is above 50 Gy, over a predetermined number of sessions, wherein (xv) the nasal tube is a nasoduodanal tube, or nasojejunal tube, (xvi) the distal end of the nasal tube is operable to direct the composition comprising the cytoprotectant pro-drug or the drug composition to a predetermined location on the wall of the at least one of: the duodenum, and the jejunum, wherein (xvii) the cytoprotectant pro-drug or the drug composition comprises: ergotamine, amifostine, Amifostine thiol-amifostine, 2-[(3-Aminopropyl)amino]ethanethiol dihydrochloride (hereinafter WR-1065), pyridoxine, S-2-(3-amino propylamino)ethyl dihydrogen phosphorothioate, its active 2-[(3-Aminopropyl)amino]ethanethiol dihydrochloride (WR-1065), their pharmaceutically accepted salt, or a cytoprotectant pro-drug or drug composition comprising one or more of the foregoing, (xviii) the bio-adhesive composition is comprised of hydroxylpropyl cellulose (HPC), hydroxypropylmethylcellulose (HPMC), Hypromellose, starch, polyvinylpyrollidone (PVP), xanthan gum, or a composition comprising one or more of the foregoing, wherein (xix) the organ or the tissue sought to be irradiated is the pancreas, wherein (xx) the fractionated stereotactic body radiation is configured to expose the pancreas to a total radiation dose of between about 50 Gy and about 208 Gy, and (xxi) the composition comprising the cytoprotectant pro-drug or the drug composition is configured to gel in-situ triggered by pH change, temperature change, ionic concentration change, enzymatic activity or a combination thereof.

While in the foregoing specification the methods of use have been described in relation to certain preferred exemplary implementations, and many details are set forth for purpose of illustration, it will be apparent to those skilled in the art that the disclosure is susceptible to additional exemplary implementations and that certain of the details described in this specification and as are more fully delineated in the following claims can be varied considerably without departing from the basic principles of this invention.

What is claimed:

1. A method of protecting a portion of the small intestine from radiation damage during radiation therapy in a subject in need thereof, using a nasal tube having a proximal end and a distal end, the method comprising:
a) inserting the distal end of the nasal tube to a location in the portion of the small intestine adjacent to an organ or a tissue sought to be irradiated;
b) validating the location of the distal end;
c) administering to the portion of the small intestine an effective amount of a composition comprising a cytoprotectant pro-drug or a drug composition, wherein the cytoprotectant pro-drug or drug composition is configured to accrete in a wall of the portion of the small intestine adjacent to the irradiated organ or tissue; and
d) exposing the organ or the tissue sought to be irradiated to ablative radiation.

2. The method of claim 1, wherein the nasal tube is a nasoduodanal tube, or nasojejunal tube.

3. The method of claim 1, wherein the distal end of the nasal tube is operable to direct the composition comprising the cytoprotectant pro-drug or the drug composition to a predetermined location on the wall of the portion of the small intestine adjacent to the irradiated organ or tissue.

4. The method of claim 1, wherein the cytoprotectant pro-drug or the drug composition comprises at least one of: ergotamine, amifostine, Amifostine thiol-amifostine, 2-[(3-Aminopropyl)amino]ethanethiol dihydrochloride (hereinafter WR-1065) and pyridoxine.

5. The method of claim 4, wherein the cytoprotectant pro-drug or a drug composition comprises S-2-(3-amino propylamino)ethyl dihydrogen phosphorothioate, its active 2-[(3-Aminopropyl)amino]ethanethiol dihydrochloride (WR-1065), or their pharmaceutically accepted salt.

6. The method of claim 1, wherein the distal end of the nasal tube is further coated with a radio-opaque composition and the step of validating the location of the distal end further comprises imaging the expected location.

7. The method of claim 6, further comprising adjusting the location of the distal end prior to administering the composition comprising the cytoprotectant pro-drug or the drug composition.

8. The method of claim 1, wherein the composition comprising the cytoprotectant pro-drug further comprises a bio-adhesive composition.

9. The method of claim 8, wherein the bio-adhesive composition is comprised of hydroxylpropyl cellulose (HPC), hydroxypropylmethylcellulose (HPMC), Hypromellose, starch, polyvinylpyrollidone (PVP), xanthan gum, or a composition comprising one or more of the foregoing.

10. The method of claim 1, wherein the cytoprotectant pro-drug or the drug composition has a viscosity of between about 25 centipoise (cP) and about 1500 cP.

11. The method of claim 1, wherein the organ or tissue sought to be protected is a duodenum, a jejunum, a large intestine, a rectum, an esophagus, or a small intestine.

12. The method of claim 11, wherein the organ or the tissue sought to be irradiated is a pancreas, a uterus, a prostate, or a bladder.

13. The method of claim 12, wherein the organ or tissue sought to be protected is the duodenum, or the jejunum.

14. The method of claim 13, wherein the organ or the tissue sought to be irradiated is the pancreas.

15. The method of claim 1, wherein the step of exposing the organ or the tissue sought to be irradiated to ablative radiation comprises: using fractionated stereotactic body radiation therapy, exposing the organ or the tissue sought to be irradiated to between 1 and about 5 irradiation fractions.

16. The method of claim 15, wherein the fractionated stereotactic body radiation therapy is configured to expose the organ or the tissue sought to be irradiated to a total radiation dose of between about 50 Gy and about 208 Gy.

17. The method of claim 16, wherein the radiation is administered in between 3 and 5 fractions of 10 Gy and 17 Gy per fraction, so long as the total radiation dose is above 50 Gy.

18. The method of claim 1, wherein the composition comprising the cytoprotectant pro-drug or the drug composition is configured to gel in-situ.

19. The method of claim 18, wherein the in-situ gelation is triggered by pH change, temperature change, ionic concentration change, enzymatic activity or a combination thereof.

20. The method of claim 1, wherein the bio-adhesive composition comprises Povidone K25 having a molecular weight of about 30,000 Daltons at a concentration ranging from between about 5% and about 10% (w/v).

21. The method of claim 20, wherein the cytoprotectant pro-drug comprises Amifostine at a concentration of about 150 mg/mL.

22. The method of claim 21, wherein the effective amount of the composition achieves a tissue concentration of the cytoprotectant pro-drug or its active metabolite that is at least 1.5-fold greater than a composition not containing Povidone, and wherein the tissue concentration is at least 14 μM/g within 15 to 90 minutes of administration.

23. A method of use of a nasal tube having a proximal end and a distal end for the protection of at least one of: a duodenum, and a jejunum, each from radiation damage during ablative radiation, the method comprising:
a) inserting the distal end of the nasal tube to a location in the portion of the small intestine adjacent to an organ or a tissue sought to be irradiated, wherein the distal end of the nasal tube is further coated with a radio-opaque composition;
b) using an imaging module, validating the location of the distal end;
c) through the nasal tube, administering to the portion of the small intestine an effective amount of a composition comprising:
i. a cytoprotectant pro-drug or a drug composition, wherein the cytoprotectant pro-drug or drug composition is configured to accrete in a wall of the portion of the small intestine adjacent to the irradiated organ or tissue;
ii. and a bio-adhesive composition
d) exposing the organ or the tissue sought to be irradiated to fractionated stereotactic body radiation (SBRT), of between 3 and 5 fractions of 10 Gy and 17 Gy per fraction, so long as the total radiation dose is above 50 Gy, over a predetermined number of sessions.

24. The method of claim 23, wherein the nasal tube is a nasoduodanal tube, or nasojejunal tube.

25. The method of claim 23, wherein the distal end of the nasal tube is operable to direct the composition comprising the cytoprotectant pro-drug or the drug composition to a predetermined location on the wall of the at least one of: the duodenum, and the jejunum.

26. The method of claim 23, wherein the cytoprotectant pro-drug or the drug composition comprises at least one of: ergotamine, amifostine, Amifostine thiol-amifostine, 2-[(3-Aminopropyl)amino]ethanethiol dihydrochloride (hereinafter WR-1065), pyridoxine, S-2-(3-amino propylamino)ethyl dihydrogen phosphorothioate, its active 2-[(3-Aminopropyl)amino]ethanethiol dihydrochloride (WR-1065), or their pharmaceutically accepted salt.

27. The method of claim 26, wherein the bio-adhesive composition is comprised of hydroxylpropyl cellulose (HPC), hydroxypropylmethylcellulose (HPMC), Hypromellose, starch, polyvinylpyrollidone (PVP), xanthan gum, or a composition comprising one or more of the foregoing.

28. The method of claim 23, wherein the organ or the tissue sought to be irradiated is the pancreas.

29. The method of claim 25, wherein the fractionated stereotactic body radiation is configured to expose the pancreas to a total radiation dose of between about 50 Gy and about 208 Gy.

30. The method of claim 23, wherein the composition comprising the cytoprotectant pro-drug or the drug composition is configured to gel in-situ triggered by pH change, temperature change, ionic concentration change, enzymatic activity or a combination thereof.

31. The method of claim 23, wherein the bio-adhesive composition comprises Povidone K25 having a molecular weight of about 30,000 Daltons at a concentration ranging from between about 5% and about 10% (w/v).

32. The method of claim 31, wherein the cytoprotectant pro-drug comprises Amifostine at a concentration of about 150 mg/mL.

33. The method of claim 32, wherein the effective amount of the composition achieves a tissue concentration of the cytoprotectant pro-drug or its active metabolite that is at least 1.5-fold greater than a composition not containing Povidone, and wherein the tissue concentration is at least 14 μM/g within 15 to 90 minutes of administration.
